(19) United States
(12) Patent Application Publication
(10) Pub. No.: US 2017/0281269 A1
(43) Pub. Date: Oct. 5, 2017

(54) MEDICAL INSTRUMENT

(71) Applicant: NIPPON PISTON RING CO., LTD., Saitama City (JP)

(72) Inventors: Yoshiki ISHIKAWA, Saitama City (JP); Takasumi KUBO, Saitama City (JP); Yuki KIMURA, Saitama City (JP); Hiroshi MATSUSHIMA, Saitama City (JP)

(21) Appl. No.: 15/468,205

(22) Filed: Mar. 24, 2017

(30) Foreign Application Priority Data
Mar. 31, 2016 (JP) 2016-070092

(51) Int. Cl. A61B 18/14

(52) U.S. Cl. CPC A61B 18/1492; A61B 18/1477; A61B 2018/00577

(57) ABSTRACT

There is provided a medical instrument achieving a simple and efficient configuration. The medical instrument includes: an elongated part formed in a linear or tubular shape, at least a part of which is inserted into a living body; and an alloy part provided in the elongated part and formed from an alloy containing titanium and tantalum.

### BACKGROUND OF THE INVENTION

### 1. Field of the Invention

The present invention relates to a medical instrument, at least a part of which is inserted into a living body, such as a catheter or a guide wire.

### 2. Description of the Related Art

In the medical field, various procedures have been conventionally performed by inserting a catheter having a tubular body or a guide wire having a linear body into various tubular organs of a living body. In a procedure of dilating a stenosis site in a blood vessel, for example, a flexible guide wire is first inserted into the blood vessel until the tip thereof reaches the target stenosis site. Subsequently, a balloon catheter having a balloon at its tip is inserted into the blood vessel along the guide wire until the balloon reaches the stenosis site. Thereafter, a fluid such as air is introduced into the balloon to inflate the balloon. In this manner, the stenosis site is dilated.

Such a procedure is typically performed while a radiographic image is monitored. Thus, the tip of the catheter or guide wire includes a marker formed from a radiopaque material such as platinum or a platinum alloy in order to improve a function of radiographic visualization in a radiographic image (see Patent Literature 1 or 2, for example).

The entire contents of Patent Literature 1: Japanese Patent Application Laid-Open No. 2008-110132, and Patent Literature 2: Japanese Patent No. 4940235 are incorporated herein by reference.

### SUMMARY OF THE INVENTION

However, the tip of the catheter or guide wire typically includes a structure for adjusting its strength and rigidity to improve the reachability to a target site, and a structure for performing various procedures in the target site. A marker is further added to such structures, resulting in the complicated configuration of the tip of the catheter or guide wire. Depending on the configuration of the tip, the marker may have a difficulty in being disposed at an appropriate position, or the provision of the marker may deteriorate the function that should have been exerted by the catheter or the guide wire.

The present invention has been made in view of the aforementioned problems. It is an object of the invention to provide a medical instrument achieving a simple and efficient configuration.

(1) An aspect of the present invention is a medical instrument including: an elongated part formed in a linear or tubular shape, at least a part of which is inserted into a living body; and an alloy part provided in the elongated part and formed from an alloy containing titanium and tantalum.

(2) In the medical instrument according to (1) described above, the alloy may contain tin.

(3) In the medical instrument according to (2) described above, the alloy may contain tantalum in an amount of 19 at. % or more and 27 at. % or less and tin in an amount of 2 at. % or more and 8 at. % or less relative to the whole alloy as 100 at. %, and the balance may include titanium and an unavoidable impurity.

(4) In the medical instrument according to any one of (1) to (3) described above, the alloy part may be provided to a portion to be inserted into a living body.

(5) In the medical instrument according to (4) described above, the alloy part may be provided on a tip side of the elongated part.

(6) In the medical instrument according to any one of (1) to (5) described above, the alloy part may be formed in a spiral shape.

(7) In the medical instrument according to any one of (1) to (5) described above, the alloy part may be formed in a tubular, annular, or cap shape.

(8) In the medical instrument according to any one of (1) to (5) described above, the alloy part may be formed in a tubular shape with a slit or a hole.

(9) In the medical instrument according to any one of (1) to (5) described above, the alloy part may be formed in a net or basket shape.

(10) In the medical instrument according to any one of (1) to (5) described above, the alloy part may be formed in a linear or rod shape.

(11) In the medical instrument according to any one of (1) to (5) described above, the alloy part may be formed in a flat plate or curved plate shape.

(12) In the medical instrument according to any one of (1) to (5) described above, the alloy part may be formed in a needle or needle tube shape.

(13) In the medical instrument according to any one of (1) to (5) described above, the alloy part may be formed in a columnar or block shape.

(14) In the medical instrument according to any one of (1) to (13) described above, the alloy part may provide a predetermined first function and a function of radiographic visualization.

(15) In the medical instrument according to (14) described above, the first function may be a reinforcing function for increasing a strength of the elongated part or suppressing deformation of the elongated part.

(16) In the medical instrument according to (14) described above, the first function may be a rigidity adjusting function of adjusting axial rigidity, bending rigidity, or torsional rigidity of the elongated part.

(17) In the medical instrument according to (14) described above, the first function may be a shaping function for causing the elongated part to have a predetermined bent shape.

(18) In the medical instrument according to (14) described above, the first function may be a scraping function of scraping a part of a living body or an accretion to a living body.

(19) In the medical instrument according to (14) described above, the first function may be a filter function for trapping a substance moving through a living body or filtering a fluid in a living body.

(20) In the medical instrument according to (14) described above, the first function may be an ablation function of ablating a part of a living body or an accretion to a living body.

(21) In the medical instrument according to (14) described above, the first function may be an anchoring function of anchoring to a living body.

(22) In the medical instrument according to (14) described above, the first function may be a holding function of holding a part of a living body or an accretion to a living body.

(23) In the medical instrument according to (14) described above, the first function may be a cutting function of cutting a part of a living body or an accretion to a living body.

(24) In the medical instrument according to (14) described above, the first function may be a needling function of needling into a tissue of a living body.

(25) In the medical instrument according to (14) described above, the first function may be a nozzle function for discharging a fluid into a living body.

(26) In the medical instrument according to (14) described above, the first function may be a spiral propelling function for moving the elongated part in an axial direction by a rotation of the elongated part about the axial direction.

(27) In the medical instrument according to (14) described above, the first function may be a passive function for moving the elongated part by a peristalsis of a living body.

(28) In the medical instrument according to (14) described above, the first function may be an electrode function for passing an electric current through a living body or generating an electric field in a living body.

(29) In the medical instrument according to (14) described above, the first function may be a length measurement function of measuring a length in a living body.

The medical instrument of the present invention can provide an advantageous effect of achieving a simple and efficient configuration.

### BRIEF DESCRIPTION OF THE DRAWINGS

FIG. 1 is a schematic diagram showing a medical instrument according to an embodiment of the present invention;

FIGS. 2A to 2J are schematic diagrams showing specific examples of a first function provided by an alloy part and the shape of the alloy part;

FIGS. 3A to 3F are schematic diagrams showing specific examples of the first function provided by the alloy part and the shape of the alloy part;

FIGS. 4A to 4E are schematic diagrams showing specific examples of the first function provided by the alloy part and the shape of the alloy part;

FIGS. 5A to 5J are schematic diagrams showing specific examples of the first function provided by the alloy part and the shape of the alloy part;

FIGS. 6A to 6H are schematic diagrams showing specific examples of the first function provided by the alloy part and the shape of the alloy part;

FIGS. 7A to 7D are schematic diagrams showing specific examples of the first function provided by the alloy part and the shape of the alloy part;

FIGS. 8A to 8D are schematic diagrams showing specific examples of the first function provided by the alloy part and the shape of the alloy part;

FIGS. 9A to 9D are schematic diagrams showing specific examples of the first function provided by the alloy part and the shape of the alloy part;

FIGS. 10A and 10B are schematic diagrams showing specific examples of the first function provided by the alloy part and the shape of the alloy part; and

FIGS. 11A to 11C are schematic diagrams showing specific examples of the first function provided by the alloy part and the shape of the alloy part.

### DETAILED DESCRIPTION OF THE PREFERRED EMBODIMENTS

An embodiment of the present invention will now be described below with reference to the accompanying drawings. Note that the drawings include unshown or simplified parts for ease of comprehension. Note also that the shapes or dimension ratios of elements in the drawings are not necessarily accurate.

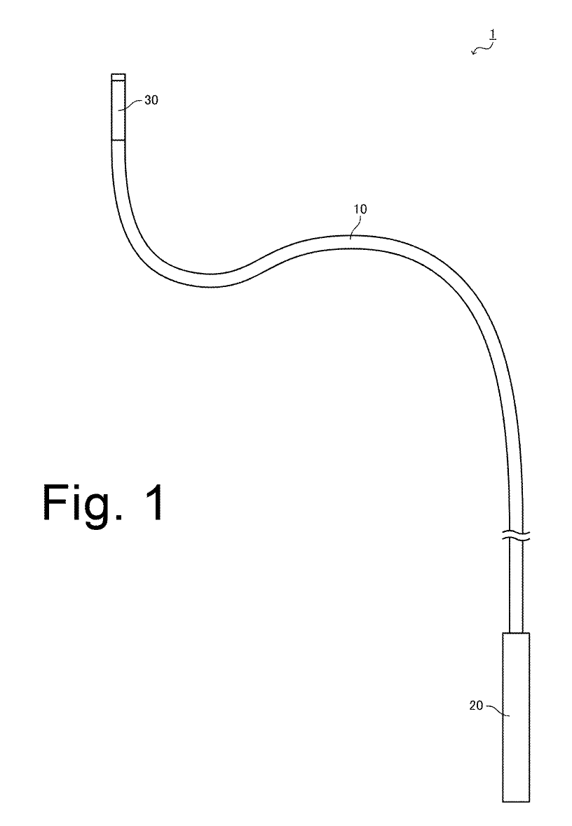

FIG. 1 is a schematic diagram showing a medical instrument 1 of the present embodiment. At least a part of the medical instrument 1 is inserted into a tubular organ such as a blood vessel, the ureter, the bile duct, the trachea, and the intestine in a living body such as a human body to perform various procedures such as examinations, diagnoses, and treatment. As shown in FIG. 1, the medical instrument 1 includes: an elongated part 10, at least a part of which is inserted into a living body; an operation part 20 provided on the hand side of the elongated part 10; and an alloy part 30 provided on the tip side of the elongated part 10.

The elongated part 10 is an elongated member that has appropriate flexibility, and is inserted into a living body from its tip side. The elongated part 10 may be configured in the form of a hollow tube or a solid line. The elongated part 10 configured in a tubular shape may include a plurality of lumens (axially continuous passages). The linearly-configured elongated part 10 may be formed by a single line or by a plurality of lines such as strands. The elongated part 10 may include a tubular body through which a linear body for operating a tip part, for example, is inserted. In other words, the medical instrument 1 of the present embodiment includes both a variety of catheters and guide wires.

The material of the elongated part 10 is not limited to any particular material. Appropriate materials, such as various resins, various metals, alloys, or composite materials thereof, may be employed in accordance with the intended purpose or function. Although the cross-sectional shape of the elongated part 10 is not limited to any particular shape, at least the outer peripheral shape thereof is preferably a generally circular or generally elliptical shape in order to reduce invasiveness to a living body. The axial dimension (length) and across-the-width dimension (outer diameter) of the elongated part 10 are not limited to particular values. Appropriate dimensions are set in accordance with the type of the tubular organ into which the elongated part 10 is inserted or the location of the target site, for example.

The operation part 20 is a part not to be inserted into a living body. The operation part 20 is a part grasped by a user of the medical instrument 1, such as a doctor, to operate the medical instrument 1. The operation part 20 is formed in a shape suitable for operations such as axially pushing or withdrawing the medical instrument 1 or rotating the medical instrument 1 about the axial direction. When the medical instrument 1 is a catheter, the operation part 20 includes an insertion and removal opening through which various guide wires are inserted or removed.

The alloy part 30 is formed from a titanium-tantalum (Ti—Ta) alloy containing at least titanium (Ti) and tantalum (Ta). The alloy part 30 provides a predetermined first function and a function of radiographic visualization. Specifically, the alloy part 30 provides the first function such as a function for adjusting the strength or rigidity of the elongated part 10 as well as a function as a marker that is shown in a radiographic image.

In the conventional techniques, the part for adjusting the strength or rigidity of the elongated part 10 is formed, for example, from stainless steel such as SUS316L or a nickel-titanium (Ni—Ti) alloy, which is a superelastic alloy, in order to obtain required mechanical properties (tensile strength, Young's modulus, and elastic limit, for example). These materials, however, have low X-ray absorptivities. Consequently, such materials have difficulty in being shown clearly in a radiographic image, thus creating a need to provide a marker separately. Moreover, while conventional markers are typically formed from platinum or a platinum alloy, these materials have poor mechanical properties. Thus, it is difficult for such a marker to provide a function other than the function of radiographic visualization.

The titanium-tantalum alloy, in contrast, has a high X-ray absorptivity (radiopaque), due to the inclusion of tantalum having a large atomic weight, while having a tensile strength and a Young's modulus equivalent to those of the nickel-titanium alloy, which is a superelastic alloy. Thus, the marker can be omitted by allowing the alloy part 30 formed from the titanium-tantalum alloy to take the role conventionally assumed by the stainless steel or the nickel-titanium alloy in the elongated part 10. Moreover, by allowing the alloy part 30 to constitute a marker, another function such as a reinforcing function or a rigidity adjusting function can be imparted to the marker.

In other words, the provision of the alloy part 30 in the medical instrument 1 allows for the simple and efficient configuration of the elongated part 10. Consequently, the tip of the elongated part 10 can be formed in a more compact manner, and the bendability of the tip of the elongated part 10 can be increased, for example. Thus improving the functions and versatility of the medical instrument 1. Additionally, the cost of the medical instrument 1 can be reduced and the productivity of the medical instrument 1 can be improved.

Furthermore, the titanium-tantalum alloy has an elastic limit suitably lower than that of the nickel-titanium alloy. Thus, functions, which are difficult to achieve with the conventional nickel-titanium alloy, can be imparted to the elongated part 10, e.g., doctors can shape the tip of a guide wire with their own fingers in accordance with bifurcation of a blood vessel.

The alloy constituting the alloy part 30 is not limited to any particular alloy as long as the alloy contains at least titanium and tantalum. The alloy may contain an element other than titanium and tantalum. For example, the alloy constituting the alloy part 30 may contain tin (Sn) in addition to titanium and tantalum. In such a case, more preferable mechanical properties can be obtained.

More specifically, where the whole alloy is defined to be 100 atom percent (at. %), the alloy constituting the alloy part 30 preferably contains tantalum in an amount of 19 at. % or more and 27 at. % or less and tin in an amount of 2 at. % or more and 8 at. % or less, and the balance includes titanium and unavoidable impurities. Such an alloy can obtain not only more preferable mechanical properties, i.e., a high tensile strength, a low Young's modulus, and a suitable elastic limit, but also a high affinity for living bodies.

The position at which the alloy part 30 is provided is not limited to any particular position. In view of providing the predetermined first function given by the alloy part 30 and providing the function of radiographic visualization, however, the alloy part 30 is preferably provided to a portion to be inserted into a living body. Moreover, considering that various procedures by a catheter or a guide wire are mainly performed with the tip of the catheter, the alloy part 30 is preferably provided on the tip side of the elongated part 10.

The alloy part 30 may be provided so as to be exposed to the exterior of the elongated part 10. Alternatively, the alloy part 30 may be provided so as to be accommodated or embedded in the elongated part 10. The alloy part 30 may be provided in a partial area of the elongated part 10 in the axial direction or provided over the entire area of the elongated part 10 in the axial direction. The alloy part 30 may constitute the entire elongated part 10. In such a case, the operation part 20 may also be constituted by the alloy part 30. Examples of the medical instrument 1 include guide wires made of a titanium-tantalum alloy, metal (alloy) catheters, and syringe needles. Needless to say, a plurality of alloy parts 30 may be provided to the elongated part 10.

The shape of the alloy part 30 is not limited to any particular shape. Various shapes, such as a spiral (coil) shape, a tubular shape, an annular (ring) shape, a cap shape, a net (mesh) shape, a basket shape, a linear shape, a rod shape, a flat plate shape, a curved plate shape, a needle shape, a needle tube shape, a columnar shape, or a block shape, can be employed in accordance with the first function provided by the alloy part 30. Since the provision of the alloy part 30 as described above can omit the marker, the shape of the alloy part 30 can be optimized more than the conventional techniques. Alternatively, the shape of the alloy part 30 may be set on the basis of the shape shown in radiographic images.

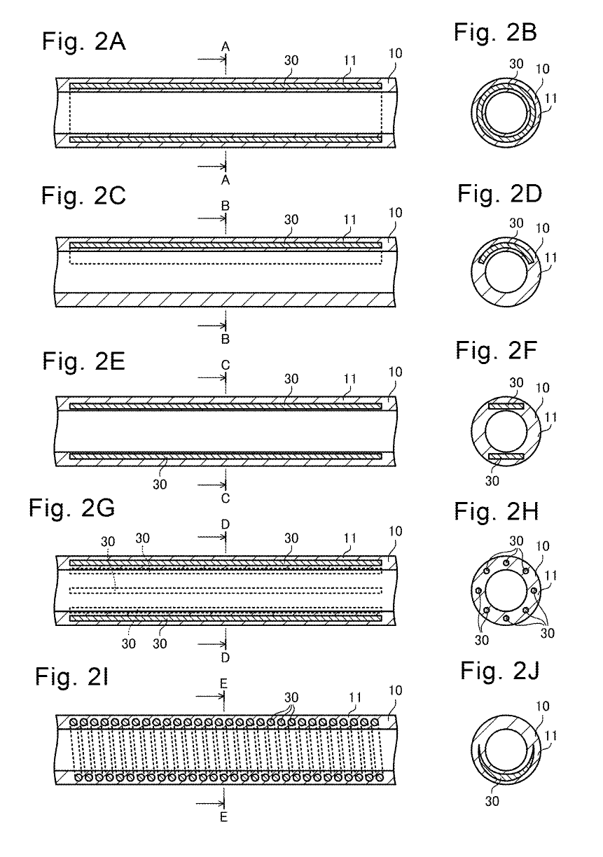

Specific examples of the first function provided by the alloy part 30 and the shape of the alloy part 30 will be described next. FIGS. 2A to 8D are schematic diagrams showing specific examples of the first function provided by the alloy part 30 and the shape of the alloy part 30.

FIGS. 2A to 2J are schematic cross-sectional views showing examples when the medical instrument 1 is a catheter and the first function is a reinforcing function for increasing the strength of the elongated part 10 or suppressing the deformation of the elongated part 10. Note that FIGS. 2A, 2C, 2E, 2G, and 2I show cross sections along the axial direction of the elongated part 10. FIG. 2B shows a cross section taken along line A-A of FIG. 2A, FIG. 2D shows a cross section taken along line B-B of FIG. 2C, FIG. 2F shows a cross section taken along line C-C of FIG. 2E, FIG. 2H shows a cross section taken along line D-D of FIG. 2G, and FIG. 2J shows a cross section taken along line E-E of FIG. 2I.

The strength of the elongated part 10 can be increased and a deformation such as the elongation and contraction, bending, or torsion of the elongated part 10 can be suppressed by embedding the alloy part 30 having an appropriate shape in a peripheral wall 11 of the elongated part 10 formed from an appropriate resin, for example, as shown in the figures. Consequently, the kink of the elongated part 10 can be prevented from occurring, or the torque transmission characteristics of the elongated part 10 can be enhanced to improve the operability of the elongated part 10, for example.

Examples of the shape of the alloy part 30 in such a case include a tubular (cylindrical) shape as shown in FIGS. 2A and 2B, a curved plate shape along the peripheral wall 11 as shown in FIGS. 2C and 2D, a flat plate shape as shown in FIGS. 2E and 2F, a rod shape as shown in FIGS. 2G and 2H, and a spiral shape as shown in FIGS. 2I and 2J. Other shapes, however, may be employed. Although FIGS. 2A to 2J show cases where the alloy part 30 is provided in a partial area of the elongated part 10 in the axial direction, the alloy part 30 may, of course, be provided over the entire area of the elongated part 10.

In this case, a plurality of alloy parts 30 having the same shape may be provided as shown in FIGS. 2E and 2F and FIGS. 2G and 2H, or a combination of a plurality of alloy parts 30 having different shapes may be provided. Alternatively, the ease of deformation in the elongated part 10 may be varied according to positions in the axial direction by arranging the alloy parts 30 having different shapes along the axial direction of the elongated part 10 or by changing the pitch of the spiral alloy part 30 shown in FIGS. 2I and 2J, for example.

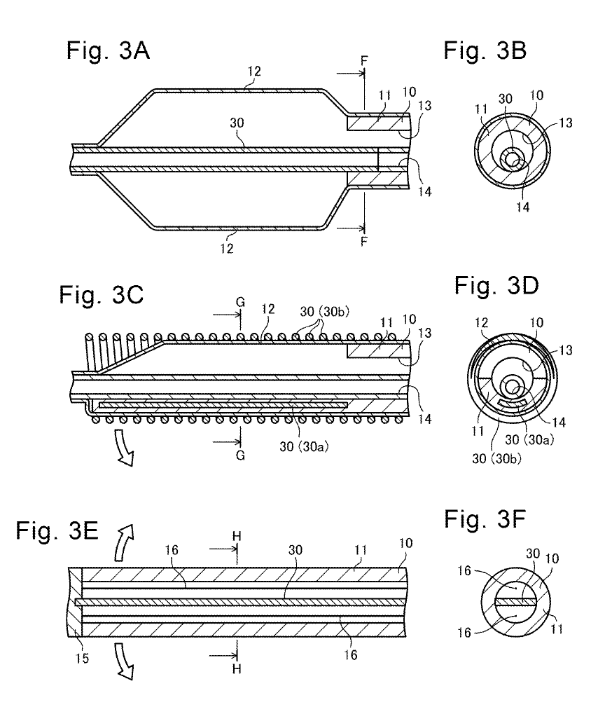

FIGS. 3A and 3B are schematic cross-sectional views showing an example when the medical instrument 1 is a balloon catheter and the first function is a reinforcing function for suppressing the axial deformation of the elongated part 10. Note that FIG. 3A shows a cross section along the axial direction of the elongated part 10. FIG. 3B shows a cross section taken along line F-F of FIG. 3A.

The elongated part 10 in this example includes: a balloon 12 provided at a tip thereof; an inflation lumen 13 through which a fluid for inflating the balloon 12 passes; and a guide wire lumen 14 through which a guide wire is inserted. The alloy part 30 is formed in a tubular shape so that the interior thereof is in communication with the guide wire lumen 14. The alloy part 30 is disposed at the position corresponding to the balloon 12 so as to support the balloon 12. Additionally, the alloy part 30 is provided so that the tip thereof protrudes toward the tip side (the left side in the figure) of the elongated part 10 beyond the balloon 12.

The provision of such an alloy part 30 can suppress the axial deformation of the tip of the elongated part 10 at which the balloon 12 is provided and thus enhance the pushability. Since the titanium-tantalum alloy has a low Young's modulus, the pushability can be enhanced while maintaining the flexibility for bending.

The balloon 12 inflates not only in the radial direction but also in the axial direction during the inflation thereof. The support of the balloon 12 by the alloy part 30, however, allows the axial dimension of the balloon 12 to restore the dimension before the inflation by the restoring force of elastic deformation of the alloy part 30 when the once-inflated balloon 12 is deflated. More specifically, when the balloon 12 is supported by a supporting member formed from a resin, for example, the inflation of the balloon 12 may cause the permanent deformation (plastic deformation) of the supporting member, thus failing to restore the pre-inflation state of the balloon 12 when deflated. This may make the movement of the elongated part 10 difficult. This example, however, can prevent such a problem.

Moreover, since the torsion or kink of the balloon 12 part can be suppressed upon the inflation or deflation of the balloon 12, reliability in a severe use environment such as intra-aortic balloon pumping (IABP) can be increased.

Also in this case, various shapes, without being limited to any particular shape, can be employed as the shape of the alloy part 30. The alloy part 30 may be disposed so as to be embedded in the peripheral wall 11 of the elongated part 10.

FIGS. 3C and 3D are schematic cross-sectional views showing an example when the medical instrument 1 is a catheter configured so that a tip thereof is bent by the inflation of the balloon 12, and the first function is a reinforcing function for partially suppressing the axial elongation of the elongated part 10 and a reinforcing function for suppressing the radial inflation of the balloon 12. Note that FIG. 3C shows a cross section along the axial direction of the elongated part 10. FIG. 3D shows a cross section taken along line G-G of FIG. 3C.

In this example, the balloon 12 is configured so that only a predetermined side (the upper side in the figure) of the elongated part 10 in the radial direction is inflated. An alloy part 30a formed in a curved plate shape is provided on the other side (the lower side in the figure) in order to partially suppress the axial elongation of the elongated part 10 in a part of the elongated part 10 in the circumferential direction thereof. Furthermore, in this example, a spiral alloy part 30b is provided so as to cover the outer periphery of the elongated part 10 including the balloon 12. This suppresses the radial inflation of the balloon 12.

Such a configuration allows the balloon 12 to inflate appropriately only in the generally axial direction. Such a configuration can also maintain the flexibility for the bending of the elongated part 10 while allowing the tip side (the left side in the figure) of the elongated part 10 to bend toward the predetermined other side (the lower side in the figure) in the radial direction. Note that the shape of the alloy part 30a is not limited to any particular shape. A rod shape or a flat plate shape, for example, may be employed. Also, the shape of the alloy part 30b is not limited to the spiral shape. For example, a plurality of tubular or annular alloy parts 30b may be provided.

Alternatively, a member formed from a material other than the titanium-tantalum alloy may be provided instead of the alloy part 30a or the alloy part 30b. In other words, only one of the alloy part 30a that provides the reinforcing function for partially suppressing the axial elongation of the elongated part 10 and the alloy part 30b that provides the reinforcing function for suppressing the radial inflation of the balloon 12 may be provided.

FIGS. 3E and 3F are schematic cross-sectional views showing an example when the medical instrument 1 is a steering catheter and the first function is a reinforcing function for suppressing the axial deformation of the elongated part 10. Note that FIG. 3E shows a cross section along the axial direction of the elongated part 10. FIG. 3F shows a cross section taken along line H-H of FIG. 3E.

In this example, the alloy part 30 is formed in a generally flat plate shape and disposed approximately at a center position inside the elongated part 10 along the axial direction of the elongated part 10. The tip-side end of the alloy part 30 is connected to a tip member 15 provided at the tip of the elongated part 10. Two operation wires 16 passing through both sides of the alloy part 30 in the thickness direction thereof are connected to the tip member 15. In other words, the elongated part 10 in this example is configured to be bendable by pulling one of the two operation wires 16 toward the hand side. Since the titanium-tantalum alloy has a low Young's modulus as mentioned above, such a configuration allows high pushability, flexible bendability, and smooth operability to be achieved simultaneously.

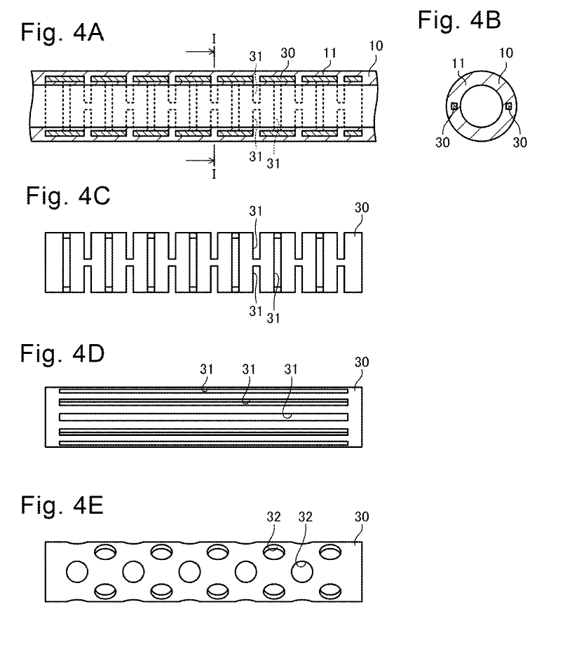

FIGS. 4A and 4B are schematic cross-sectional views showing an example when the medical instrument 1 is a catheter and the first function is a rigidity adjusting function of adjusting the axial rigidity, bending rigidity, or torsional rigidity of the elongated part 10. Note that FIG. 4A shows a cross section along the axial direction of the elongated part 10. FIG. 4B shows a cross section taken along line I-I of FIG. 4A. FIGS. 4C to 4E are schematic diagrams showing examples of the shape of the alloy part 30 in such an example.

The axial rigidity, bending rigidity, or torsional rigidity of the elongated part 10 can be adjusted by embedding the tubular alloy part 30 provided with appropriate slits 31 or holes 32 in the peripheral wall 11 of the elongated part 10 formed from an appropriate resin, for example, as shown in the figures. In other words, the ease of elastic deformation such as the elongation and contraction, bending, or torsion of the elongated part 10 can be adjusted individually and more finely by appropriately setting the shape of the slit 31 or the hole 32 and the arrangement thereof in the alloy part 30.

In this case, the slit 31 may be formed along the circumferential direction as shown in FIG. 4C, along the axial direction as shown in FIG. 4D, or along any other direction. The slit 31 is not limited to the linearly formed slit. For example, the slit 31 may be formed in a curved or zigzag shape.

Also, the hole 32 may have any shape without being limited to the circular shape shown in FIG. 4E. Needless to say, the arrangement of the holes 32 is not limited to any particular arrangement. Alternatively, a combination of the slits 31 and the holes 32 may be provided in the alloy part 30. Instead of the tubular alloy part 30, the curved plate-shaped or flat plate-shaped alloy part 30 provided with the slits 31 or the holes 32 may be provided. Alternatively, different kinds of alloy parts 30 may be combined, or the shapes of the slits 31 or the holes 32, for example, may be varied according to their positions.

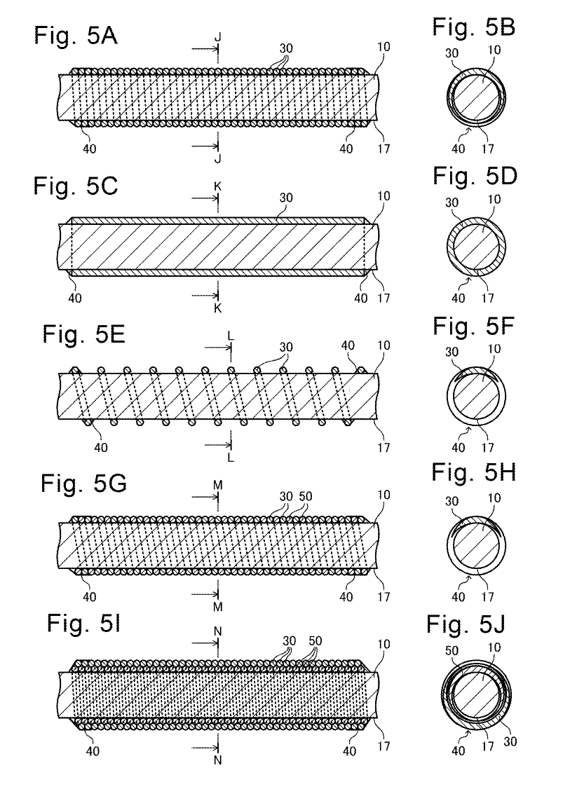

FIGS. 5A to 5J are schematic cross-sectional views showing examples when the medical instrument 1 is a guide wire and the first function is a reinforcing function for increasing the strength of the elongated part 10 or suppressing the deformation of the elongated part 10. Note that FIGS. 5A, 5C, 5E, 5G, and 5I show cross sections along the axial direction of the elongated part 10. FIG. 5B shows a cross section taken along line J-J of FIG. 5A, FIG. 5D shows a cross section taken along line K-K of FIG. 5C, FIG. 5F shows a cross section taken along line L-L of FIG. 5E, FIG. 5H shows a cross section taken along line M-M of FIG. 5G, and FIG. 5J shows a cross section taken along line N-N of FIG. 5I.

In the example shown in FIGS. 5A and 5B, the alloy part 30 having a spiral shape is disposed so as to cover an outer peripheral surface 17 of the elongated part 10 formed from an appropriate metal or alloy, for example. The both ends of the alloy part 30 in the axial direction are each fixed to the elongated part 10 with an appropriate brazing material 40. In the example shown in FIGS. 5C and 5D, the alloy part 30 having a tubular shape is disposed so as to cover the outer peripheral surface 17 of the elongated part 10 formed from an appropriate metal or alloy, for example. The both ends of the alloy part 30 in the axial direction are each fixed to the elongated part 10 with the appropriate brazing material 40.

The strength of the elongated part 10 can be increased and a deformation such as the elongation and contraction, bending, or torsion of the elongated part 10 can be suppressed by providing the alloy part 30 so as to cover the outer peripheral surface 17 of the elongated part 10 formed from an appropriate metal or alloy, for example, as described above. Consequently, the kink of the elongated part 10 can be prevented from occurring, or the torque transmission characteristics of the elongated part 10 can be enhanced to improve the operability of the elongated part 10, for example.

Also in this case, the shape of the alloy part 30 is not limited to any particular shape. For example, the alloy part 30 formed in a curved plate shape or a rod shape may be fixed to the outer peripheral surface 17. Alternatively, a combination of a plurality of alloy parts 30 having different shapes may be provided, or the pitch of the spiral alloy part 30 may be varied. Alternatively, the elongated part 10 and the alloy part 30 may be covered with an appropriate resin, for example. The method for fixing the alloy part 30 may be any known method other than the brazing, such as engagement or welding.

FIGS. 5E and 5F show a case where the alloy part 30 having a spiral shape is wound around the elongated part 10 with the winds not being in close contact with one another but having some distance between the winds. The thus configured alloy part 30 can achieve a state as if a thread had been formed on the outer peripheral surface 17 of the elongated part 10. Thus, the elongated part 10 can be moved in the axial direction by rotating the elongated part 10 about the axial direction. In other words, the alloy part 30 in this example provides, as the first function, the aforementioned reinforcing function and a spiral propelling function.

FIGS. 5G and 5H show a case where a spiral auxiliary member 50 formed from a material different from that of the alloy part 30, such as a nickel-titanium alloy, is wound around the elongated part 10 so that the spiral auxiliary member 50 and the spiral alloy part 30 together form a double-threaded spiral. FIGS. 5I and 5J show a case where the spiral auxiliary member 50 formed from a different material is wound around the elongated part 10, and the spiral alloy part 30 is further wound therearound.

Depending on the intended purpose, use environment, etc., of the medical instrument 1, an appropriate combination of such an auxiliary member 50 formed from a different material and the alloy part 30 can impart more preferable characteristics to the elongated part 10. Also in this case, the shapes of the alloy part 30 and the auxiliary member 50 are, of course, not limited to particular shapes. For example, a combination of the spiral alloy part 30 and a tubular auxiliary member 50 may be used.

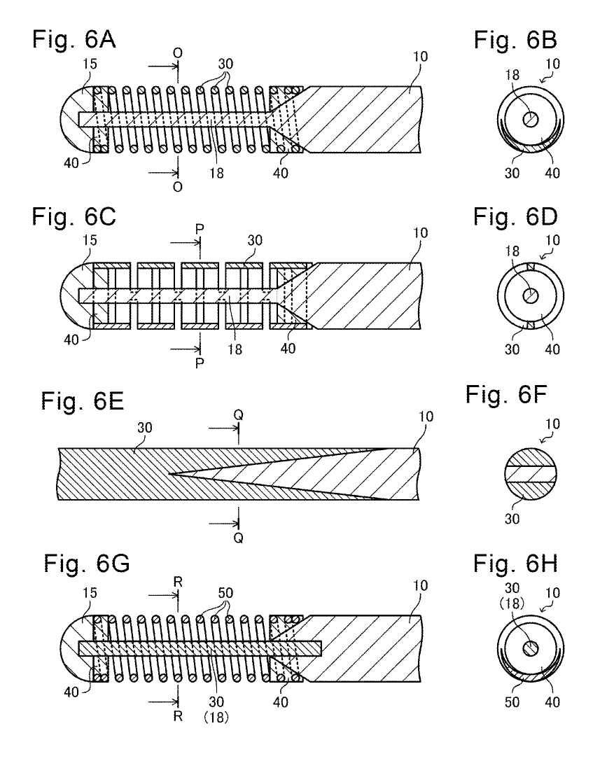

FIGS. 6A to 6H are schematic cross-sectional views each showing an example when the medical instrument 1 is a guide wire and the first function is a rigidity adjusting function of adjusting the axial rigidity, bending rigidity, or torsional rigidity of the elongated part 10. Note that FIGS. 6A, 6C, 6E, and 6G show cross sections along the axial direction of the elongated part 10. FIG. 6B shows a cross section taken along line O-O of FIG. 6A, FIG. 6D shows a cross section taken along line P-P of FIG. 6C, FIG. 6F shows a cross section taken along line Q-Q of FIG. 6E, and FIG. 6H shows a cross section taken along line R-R of FIG. 6G.

In the example shown in FIGS. 6A and 6B, a reduced diameter part 18 having a reduced outer diameter is provided on the tip side of the elongated part 10 formed from an appropriate metal or alloy, for example. The alloy part 30 having a spiral shape is disposed so as to cover the reduced diameter part 18. The both ends of the alloy part 30 in the axial direction are each fixed to the elongated part 10 by the appropriate brazing material 40. In the example shown in FIGS. 6C and 6D, the reduced diameter part 18 having a reduced outer diameter is provided on the tip side of the elongated part 10 formed from an appropriate metal or alloy, for example. The alloy part 30 having a tubular shape and provided with the slits 31 is disposed so as to cover the reduced diameter part 18. The both ends of the alloy part 30 in the axial direction are each fixed to the elongated part 10 by the appropriate brazing material 40. In these examples, the tip member 15 having a generally hemispherical shape is provided at the tip of the elongated part 10, and the tip-side end of the alloy part 30 is fixed to the elongated part 10 via the tip member 15.

Such a combination of the reduced diameter part 18 and the alloy part 30 disposed so as to cover the reduced diameter part 18 from the outer peripheral side thereof can reduce the bending rigidity of the elongated part 10 sufficiently and thus increase the bendability thereof. Consequently, the elongated part 10 can be easily caused to enter an intricate site such as a blood vessel. Moreover, the bending rigidity of the elongated part 10, for example, can be appropriately set by adjusting the outer diameter or cross-sectional shape of the reduced diameter part 18 and the pitch of the spiral alloy part 30 or the shape of the slit 31.

Also in this case, a combination of a plurality of alloy parts 30 having different shapes may be provided, or the pitch of the spiral alloy part 30 or the shape of the slit 31, for example, may be varied. The tubular alloy part 30 may be provided with the holes 32. Moreover, the elongated part 10 and the alloy part 30 may be covered with an appropriate resin, for example, or an appropriate resin, for example, may be filled between the reduced diameter part 18 and the alloy part 30.

FIGS. 6E and 6F show an example when the alloy part 30 is joined continuously to the elongated part 10 formed from an appropriate metal or alloy, for example. In this example, an end of the elongated part 10 having a linear shape is formed in a wedge shape. The alloy part 30 having a linear shape and provided with a V-groove at an end thereof is combined with and welded, for example, to the wedge-shaped end of the elongated part 10 so as to extend the elongated part 10 continuously by the alloy part 30. With such a configuration, the mechanical property of the elongated part 10 having a linear body can be partially changed. Moreover, by utilizing the suitably low elastic limit of the titanium-tantalum alloy, the tip of the elongated part 10 can be configured to be shapable by bending with fingers, for example.

Various known methods may be employed as a method for joining the elongated part 10 and the alloy part 30 together. Either one of the tip side and the hand side of the elongated part 10 can be extended by joining the alloy part 30 thereto. Alternatively, linear bodies formed from a different material may be joined to the both ends of the alloy part 30 having a linear shape so that the alloy part 30 is provided in a middle part of the elongated part 10 in the axial direction. Alternatively, outer diameters (across-the-width dimension) or cross-sectional shapes may be varied between the alloy part 30 and the other part.

FIGS. 6G and 6H show a case where the alloy part 30 having a small diameter is joined to the tip of the elongated part 10 formed from an appropriate metal or alloy, for example, to form the reduced diameter part 18 and the auxiliary member 50 having a spiral shape and formed from a material different from that of the alloy part 30 is disposed so as to cover the reduced diameter part 18. As with the example shown in FIGS. 6A and 6B, the bendability of the elongated part 10 can be increased also in this case. Moreover, the bending rigidity of the elongated part 10 can be appropriately set by adjusting the outer diameter or cross-sectional shape of the alloy part 30 (the reduced diameter part 18) and the material or pitch of the auxiliary member 50, for example.

Also in this case, the cross-sectional shape of the alloy part 30 (the reduced diameter part 18) is not limited to any particular shape. Alternatively, the alloy part 30 may be formed in a tubular shape. Alternatively, the auxiliary member 50 may be formed in a tubular shape having slits or holes. Alternatively, the alloy part 30 may include both of the reduced diameter part 18 and the member disposed so as to cover the reduced diameter part 18.

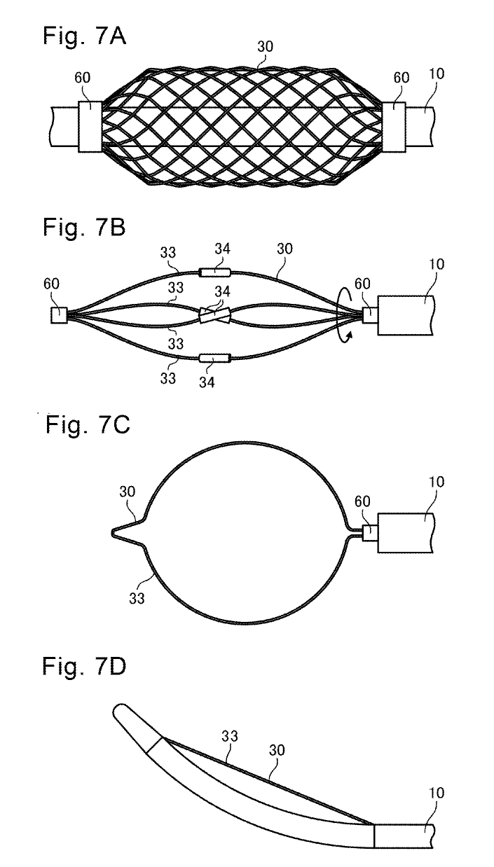

FIG. 7A is a schematic diagram showing an example when the medical instrument 1 is a scraping wire for emboli, for example, and the first function is a scraping function of scraping a part of a living body or an accretion to a living body. In this example, the alloy part 30 is formed in a net or basket shape bulging toward the outer peripheral side of the elongated part 10. The both ends of the alloy part 30 in the axial direction are each banded by a banding member 60 so as to have a reduced diameter. The both ends of the alloy part 30 are each fixed to the elongated part 10 via the banding member 60.

Since the titanium-tantalum alloy has a low Young's modulus as mentioned above, the alloy part 30 formed in a net or basket shape allows for its appropriate elastic deformation so as to be inserted into a catheter together with the elongated part 10. Additionally, the alloy part 30 can restore its original shape after the alloy part 30 is projected from the tip of the catheter. An embolus, or the like, can be scraped by reciprocating the alloy part 30 in the axial direction in a stenosis site in a blood vessel, for example.

The shape of the alloy part 30 in this case is not limited to the shape shown in FIG. 7A. Various shapes and net configurations may be employed. The alloy part 30 formed in a net or basket shape can provide, as the first function, a filter function for trapping a substance moving through a living body or filtering a fluid in a living body. For example, while an embolus in a tubular organ may be scraped, another alloy part 30 formed in a net or basket shape may be disposed in the vicinity of the alloy part used for scraping. This alloy part 30 can prevent the scraped embolus from escaping into other sites.

FIGS. 7B to 7D are schematic views each illustrating an example when the medical instrument 1 is a catheter for performing ablation with a high-frequency current and the first function is an ablation function of ablating a part of a living body or an accretion to a living body.

In the example shown in FIG. 7B, the alloy part 30 is constituted by four linear members 33 inserted through a lumen of the elongated part 10, and four electrodes 34 each provided on the linear member 33. The four linear members 33 are banded with the banding members 60 at two locations in the axial direction. The electrodes 34 are disposed between the two banding members 60. The linear members 33 are gently bent in advance between the two banding members 60. When projected from the lumen of the elongated part 10, the linear members 33 expand in a radially outer direction by the restoring force of the elastic deformation, thus disposing the electrodes 34 at predetermined positions. The ablation by the electrodes 34 is performed while the four linear members 33 are rotated.

Since the titanium-tantalum alloy has an elastic limit suitably lower than that of the nickel-titanium alloy as mentioned above, the bent shape of the linear member 33 can be adjusted with fingers. In other words, a doctor can bend the linear members 33 according to a state of an ablated site in order to adjust the arrangement of the electrodes 34 appropriately.

FIG. 7C shows a case where the alloy part 30 constitutes a high-frequency snare. The alloy part 30 in this example includes the linear member 33 having a generally loop shape with the hand side thereof being banded by the banding member 60. By drawing the alloy part 30 into the lumen of the elongated part 10, the loop has a reduced diameter. Thus, a polyp, for example, can be ablated while being strangulated by such a loop.

FIG. 7D shows a case where the alloy part 30 constitutes an incision wire of a papillotomy knife. The alloy part 30 in this example includes the linear member 33 that is inserted through the lumen of the elongated part 10, and make a part of its own tip side exposed to the outside. By pulling the alloy part 30 toward the hand side, the tip of the elongated part 10 is bent in a bow shape, thus obtaining the linearly stretched state of the alloy part 30. Thus, the alloy part 30 in such a state can ablate a part of a living body like a knife.

As mentioned above, the titanium-tantalum alloy has both of a high tensile strength and a low Young's modulus. Thus, the high-frequency snare or the incision wire of the papillotomy knife constituted by the alloy part 30 can achieve high reliability and excellent operability simultaneously. Moreover, the suitable elastic limit of the titanium-tantalum alloy allows for the fine adjustments of the shape of the high-frequency snare with fingers. Also in this case, the shape of the alloy part 30 and the number of the linear members 33 are not limited to those shown in FIGS. 7B to 7D. Needless to say, various configurations can be employed.

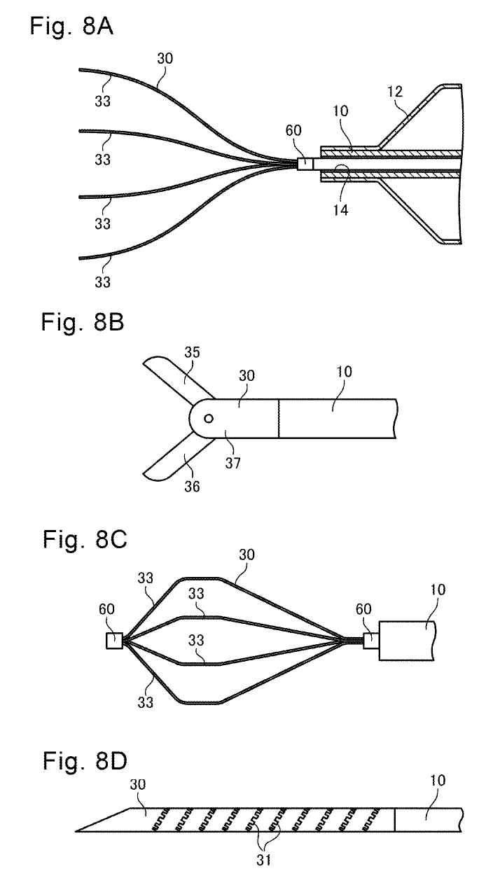

FIG. 8A is a schematic cross-sectional view showing an example when the medical instrument 1 is a balloon catheter and the first function is an anchoring function of anchoring to a living body. FIG. 8A shows a cross section along the axial direction of the elongated part 10. In this example, the alloy part 30 includes four linear members 33 inserted through the guide wire lumen 14 of the elongated part 10. The base end area of the four linear members 33 is banded by the banding member 60. The tip area of the four linear members 33 closer to the tip than the banding member 60 is gently bent in advance. In other words, when projected from the tip of the elongated part 10, the alloy part 30 expands in a radially outer direction by the restoring force of the elastic deformation, thus anchoring to a living body and positioning the balloon 12. More specifically, the anchoring of the alloy part 30 to a living body results in the positioning of the balloon 12 in the axial direction of the elongated part 10, and the radially-expanded alloy part 30 results in the positioning (centering) of the balloon 12 in the radial direction of the elongated part 10.

Depending on states of a stenosis site, the position of the balloon 12 may be displaced when the balloon 12 is inflated. The provision of the alloy part 30 for anchoring to a living body, however, can suppress the movement of the balloon 12 upon the inflation thereof and thus prevent the positional misalignment of the balloon 12. Moreover, since the linear member 33 can be bent with fingers as mentioned above, doctors can adjust the linear member 33 on their own to have an appropriate bent shape according to the size of a blood vessel, for example.

Note that any shape can be employed as the bent shape of the linear member 33 without being limited to the shape shown in FIG. 8A. Needless to say, the number of the linear members 33 is not limited to four.

FIG. 8B is a schematic view showing an example when the medical instrument 1 is a pair of scissors type catheter forceps and the first function is a holding function of holding a part of a living body or an accretion to a living body and a cutting function of cutting a part of a living body or an accretion to a living body. In this example, the alloy part 30 constitutes scissors type forceps provided at the tip of the elongated part 10. The alloy part 30 includes a pair of forceps pieces 35 and 36 capable of opening and closing, and a base 37 that swingably supports the forceps pieces 35 and 36. The forceps pieces 35 and 36 are operated via a driving wire (not shown) inserted through the lumen of the elongated part 10.

Such scissors type forceps constituted by the alloy part 30 can allow the forceps themselves to have the function of radiographic visualization. Thus, procedures such as holding or cutting a part of a living body with the scissors type forceps can be facilitated. Moreover, since there is no need to separately provide a marker, fine and complicated scissors type forceps can be configured efficiently. Note that the shape of the forceps pieces 35 and 36 is not limited to any particular shape. Various shapes can be employed according to the intended purpose. Alternatively, only the forceps pieces 35 and 36 or only the base 37 may be constituted by the alloy part 30.

FIG. 8C is a schematic view showing an example when the medical instrument 1 is a basket type catheter forcep and the first function is a holding function of holding a part of a living body or an accretion to a living body and a cutting function of cutting a part of a living body or an accretion to a living body. In this example, the alloy part 30 includes the four linear members 33 combined in a basket shape by the two banding members 60. The alloy part 30 is configured to hold a calculus, for example, by drawing the alloy part 30 containing the calculus in the basket into the lumen of the elongated part 10.

As mentioned above, the titanium-tantalum alloy has both of a high tensile strength and a low Young's modulus. Thus, such a basket forcep constituted by the alloy part 30 can achieve high reliability and excellent operability simultaneously. Moreover, the basket shape can be finely adjusted with fingers. Also in this case, the shape of the alloy part 30 and the number of the linear members 33 are not limited to any particular shape and number. Various configurations can be employed. Although its diagrammatic illustration is omitted, the banding of the tip of the alloy part 30 may be omitted to obtain claw forceps by the alloy part 30.

FIG. 8D is a schematic view showing an example when the medical instrument 1 is a biopsy needle and the first function is a cutting function of cutting a part of a living body or an accretion to a living body. In this example, the alloy part 30 is formed in a needle tube shape with a pointed tip, and the alloy part 30 is provided at the tip of the elongated part 10. The alloy part 30 is provided with appropriate slits 31 in order to increase the flexibility of the alloy part 30 for bending. The alloy part 30 is guided by a catheter or a guide wire to reach a target site. The alloy part 30 then collects a specimen by cutting a part of a living body, for example, with the pointed tip and taking in the cut part.

Since the titanium-tantalum alloy has a low Young's modulus as mentioned above, the formation of the appropriate slits 31 can make the alloy part 30 extremely flexible. Moreover, due to the high X-ray absorptivity, the entire alloy part 30 can be visually recognized during a procedure via radiographic images. Consequently, a biopsy can be performed easily and safely at a site where it has been conventionally difficult to perform a needle biopsy, such as a biopsy in a lung nodule.

The alloy part 30 in this case may be provided at the tip of the elongated part 10 formed from a different material, or the entire elongated part 10 may be constituted by the alloy part 30. The shape and arrangement of the slits 31 are not limited to any particular shape and arrangement. Depending on its intended purpose, no slits 31 may be provided.

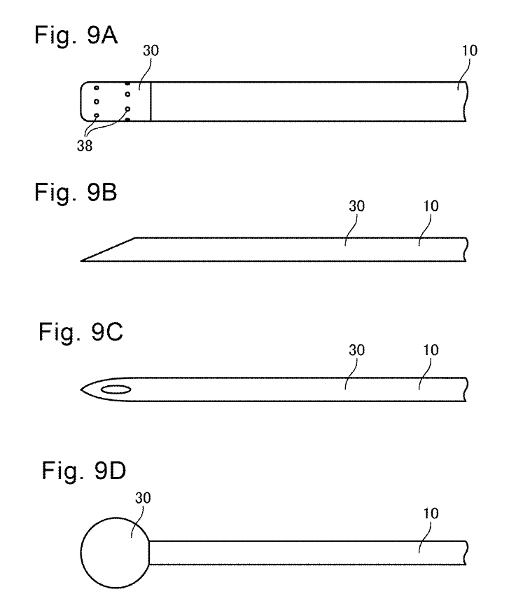

FIG. 9A is a schematic diagram showing an example when the medical instrument 1 is an irrigation catheter and the first function is a nozzle function for discharging a fluid into a living body. In this example, the alloy part 30 is formed into a block with an appropriate shape and provided at the tip of the elongated part 10. A plurality of small holes 38 in communication with the lumen of the elongated part 10 are provided on a surface of the alloy part 30. In other words, the alloy part 30 is configured to discharge various fluids (e.g., saline) supplied through the lumen of the elongated part 10 from the small holes 38.

Such an irrigation nozzle constituted by the alloy part 30 allows the nozzle itself to have the function of radiographic visualization. Thus, various procedures can be facilitated. Moreover, since there is no need to separately provide a marker, the tip of the elongated part 10 can be configured efficiently. The alloy part 30 in this case may provide an ablation function when an electric current is applied thereto.

FIGS. 9B and 9C are schematic views each showing an example when the medical instrument 1 is a syringe needle and the first function is a needling function of needling into tissues of a living body and a nozzle function for discharging a fluid into a living body. In such an example, the alloy part 30 is formed in a needle tube shape with a pointed tip, and the alloy part 30 constitutes the entire elongated part 10 that is a needle tube of a syringe needle. Note that FIG. 9C shows the case of a side-port needle.

The needle tube of the syringe needle constituted by the alloy part 30 allows the syringe needle itself to have the function of radiographic visualization. Thus, such a syringe needle can facilitate a procedure of administering a medical solution, for example, under the X-ray monitoring regardless of its simple configuration. Moreover, the excellent mechanical properties of the titanium-tantalum alloy can reduce a risk of breakage, for example. Also in this case, the shape of the alloy part 30 is not limited to any particular shape. Needless to say, various tip shapes and cross-sectional shapes of the alloy part 30 may be employed.

Although its diagrammatic illustration is omitted, the alloy part 30 formed in a solid needle shape with a pointed tip may be provided at the tip of the elongated part 10 such as a guide wire or the entire elongated part 10 may be constituted by the alloy part 30 formed in a solid needle shape with a pointed tip. In this manner, the alloy part 30 can only provide the needling function as the first function.

FIG. 9D is a schematic view showing an example when the medical instrument 1 is a guide wire or catheter for digestive organs and the first function is a passive function for moving the elongated part 10 by the peristalsis of a living body. In this example, the alloy part 30 is formed in a generally spherical shape and provided at the tip of the elongated part 10. Such a configuration causes the alloy part 30 to be transferred by the peristaltic motion of a digestive organ such as the esophagus or the intestine. Thus, the elongated part 10 can reach a predetermined site spontaneously. More specifically, the alloy part 30, which is a lump tip attached to a part of a catheter, for example, is induced in accordance with the peristaltic motion of organs such as the intestine to move in the moving direction of the peristalsis as with foods, for example. Thus, the alloy part 30 can provide a function of inducing the movement of the catheter, for example, in such a direction (the peristalsis-induced function). Moreover, the movement of the alloy part 30 can be monitored via radiographic images without separately providing a marker. Note that the shape of the alloy part 30 is not limited to the generally spherical shape. Various shapes including a polyhedron shape such as a soccer ball and an eggplant shape may be employed. The alloy part 30 may be solid or hollow.

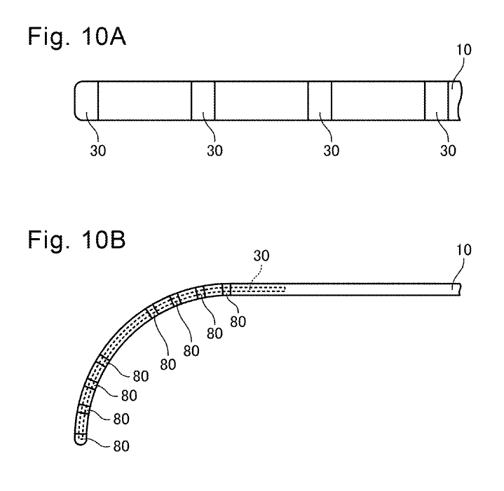

FIG. 10A is a schematic view showing an example when the medical instrument 1 is an electrode catheter for performing an electrophysiological study (EPS), for example, and the first function is an electrode function for passing an electric current through a living body or generating an electric field in a living body. In this example, the alloy part 30 is formed in an annular shape or a cap shape (at the tip). At least a part of the alloy part 30 is exposed to the exterior of the elongated part 10. A plurality of alloy parts 30 are arranged at predetermined intervals along the axial direction of the elongated part 10. The alloy parts 30 can be applied with an electric current individually via conductive wires passing through the lumen of the elongated part 10.

Such an electrode constituted by the alloy part 30 allows the position of each electrode to be checked accurately via radiographic images without separately providing a marker. Thus, the accuracy and safety of the EPS can be improved. Needless to say, the alloy part 30 in this case can also provide an ablation function.

FIG. 10B is a schematic view showing an example when the medical instrument 1 is an electrode catheter for performing an EPS, for example, and the first function is a shaping function for causing the elongated part 10 to have a predetermined bent shape. In this example, the alloy part 30 is disposed inside the tip of the elongated part 10 having a plurality of electrodes 80. The alloy part 30 is configured to keep the tip of the elongated part 10 in the predetermined bent shape by the bent shape of the alloy part 30.

Since the titanium-tantalum alloy has a low Young's modulus as mentioned above, such a configuration allows for the smooth insertion of the electrode catheter via a sheath. Moreover, since the titanium-tantalum alloy has a suitably low elastic limit, the bent shape can be finely adjusted with fingers. Furthermore, visibility near the electrodes under the X-ray monitoring can be improved.

As the shape of the alloy part 30 in this case, various shapes as shown in FIGS. 2A to 2J can be employed. The bent shape of the elongated part 10 may be an S-shape or a circular ring shape, for example, without being limited to the shape shown in FIG. 10B. Needless to say, the electrode may be constituted by the alloy part 30 in this case.

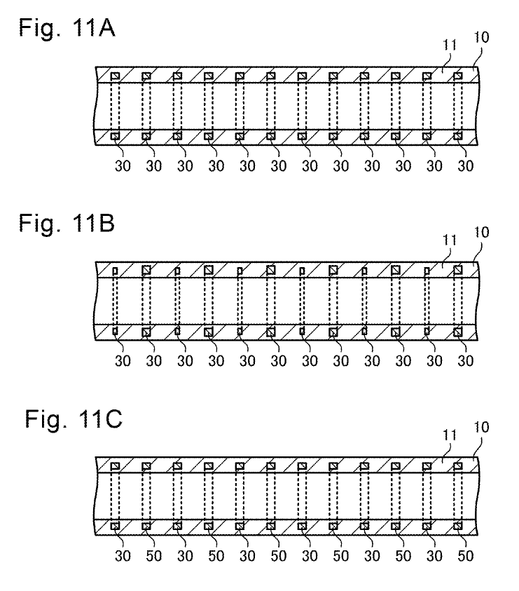

FIGS. 11A to 11C are schematic cross-sectional views each showing an example when the medical instrument 1 is a catheter and the first function is a length measurement function of measuring a length in a living body. FIGS. 11A to 11C show cross sections along the axial direction of the elongated part 10.

In the example shown in FIG. 11A, the alloy part 30 is formed in an annular shape and embedded in the peripheral wall 11. A plurality of such alloy parts 30 are arranged along the axial direction of the elongated part 10. By setting intervals between adjacent ones of the alloy parts 30 to approximately the same value, a length in a living body can be measured.

Since the titanium-tantalum alloy has a high X-ray absorptivity as mentioned above, the alloy parts 30 can be visually recognized clearly in a radiographic image. Thus, a necessary stent length, for example, can be obtained by measuring the length of a stenosis site using the alloy parts 30 shown in the radiographic image as scale marks for measuring a length.

The titanium-tantalum alloy has a high X-ray absorptivity and excellent mechanical properties. Thus, the alloy part 30 that provides the length measurement function can be configured to simultaneously provide another function such as the above-described reinforcing function or rigidity adjusting function. Similarly, the alloy part 30 that provides, for example, the reinforcing function or the rigidity adjusting function can be configured to provide the length measurement function.

Note that the shape of the alloy part 30 in this case is not limited to any particular shape. For example, the alloy part 30 may have a columnar shape, a block shape, or a spiral shape with a constant pitch. Alternatively, arrays of the alloy parts 30 may be provided so that each array has a different interval between the alloy parts 30. In this manner, more precise length measurement can be achieved.

FIG. 11B shows a case where the alloy parts 30 having different sizes are alternately arranged. The larger alloy parts 30 are used as main scale marks, whereas the smaller alloy parts 30 are used as auxiliary scale marks. Such a combination of the alloy parts 30 having different sizes or shapes, for example, can facilitate the scale reading. Thus, a length can be measured easily.

FIG. 11C shows a case where the alloy part 30 and the auxiliary member 50 formed from a different material having a high X-ray absorptivity, such as platinum or gold, are alternately arranged. Such an appropriate combination of the auxiliary member 50 having the different X-ray absorptivity and the alloy part 30 can add a contrast to the scale marks for length measurement that are shown in a radiographic image. This can facilitate the scale reading. Thus, a length can be measured easily.

As the arrangement order of the alloy parts 30 having different sizes or the arrangement order of the alloy parts 30 and the auxiliary members 50, various arrangement orders can be employed without being limited to those shown in FIGS. 11B and 11C. In the example shown in FIG. 11B, three or more different types of alloy parts 30 having different sizes or shapes, for example, may be arranged in combination with one another. In the example shown in FIG. 11C, the alloy part 30 and the auxiliary member 50 may have respective different sizes or shapes, for example. Alternatively, the alloy part 30 or the auxiliary member 50 having a different size or shape may be added.

As described above, the medical instrument 1 of the present embodiment includes: the elongated part 10 formed in a linear or tubular shape, at least a part of which is inserted into a living body; and the alloy part 30 provided in the elongated part 10 and formed from an alloy containing titanium and tantalum. Such a configuration allows for a configuration utilizing the preferable mechanical properties of the titanium-tantalum alloy. Additionally, the high X-ray absorptivity of the titanium-tantalum alloy allows for the enhanced function of radiographic visualization without separately providing a marker. Thus, the medical instrument 1 can have a simple and efficient configuration.

Preferably, the alloy constituting the alloy part 30 contains tin. This can provide more excellent mechanical properties.

Preferably, the alloy constituting the alloy part 30 contains tantalum in an amount of 19 at. % or more and 27 at. % or less and tin in an amount of 2 at. % or more and 8 at. % or less relative to the whole alloy as 100 at. %, and the balance includes titanium and unavoidable impurities. With such a configuration, a high tensile strength, a low Young's modulus, and a suitable elastic limit as well as a high affinity for living bodies can be obtained.

Preferably, the alloy part 30 is provided to a portion to be inserted into a living body. More preferably, the alloy part 30 is provided on the tip side of the elongated part 10. In this manner, the preferable characteristics of the titanium-tantalum alloy can be utilized effectively.

The alloy part 30 may be formed in a spiral shape, a tubular shape, an annular shape, a cap shape, a tubular shape with slits or holes, a net shape, a basket shape, a linear shape, a rod shape, a flat plate shape, a curved plate shape, a needle shape, a needle tube shape, a columnar shape, or a block shape. Such an alloy part 30 formed in an appropriate shape can provide various functions.

The alloy part 30 provides the predetermined first function and the function of radiographic visualization. Such an alloy part 30 providing at least two functions allows for the simple and efficient configuration of the medical instrument 1.

The first function may be the reinforcing function for increasing the strength of the elongated part 10 or suppressing the deformation of the elongated part 10, the rigidity adjusting function of adjusting the axial rigidity, bending rigidity, or torsional rigidity of the elongated part 10, the shaping function for causing the elongated part 10 to have a predetermined bent shape, the scraping function of scraping a part of a living body or an accretion to a living body, the filter function for trapping a substance moving through a living body or filtering a fluid in a living body, the ablation function of ablating a part of a living body or an accretion to a living body, the anchoring function of anchoring to a living body, the holding function of holding a part of a living body or an accretion to a living body, the cutting function of cutting a part of a living body or an accretion to a living body, the needling function of needling into tissues of a living body, the nozzle function for discharging a fluid into a living body, the spiral propelling function for moving the elongated part 10 in the axial direction by the rotation of the elongated part 10 about the axial direction, the passive function for moving the elongated part 10 by the peristalsis of a living body, the electrode function for passing an electric current through a living body or generating an electric field in a living body, the length measurement function of measuring a length in a living body, or any other function. By utilizing the preferable characteristics of the titanium-tantalum alloy, the alloy part 30 can provide various functions. Consequently, the functions and versatility of the medical instrument 1 can be improved. Additionally, the cost of the medical instrument 1 can be reduced, and the productivity thereof can be improved.

While the embodiment of the present invention has been described above, the medical instrument according to the present invention is not limited to the above-described embodiment. It will be understood that numerous modifications are possible without departing from the scope of the present invention. The functions and effects described in the above-described embodiment are merely a list of the most preferred actions and effects emanating from the present invention. Functions and effects of the present invention are not limited thereto.

The medical instrument of the present invention can be used in the medical field for humans or animals.

The entire disclosure of Japanese Patent Application No. 2016-070092 filed Mar. 31, 2016 including specification, claims, drawings, and summary are incorporated herein by reference in its entirety.

What is claimed is:

1. A medical instrument comprising:
an elongated part formed in a linear or tubular shape, at least a part of which is inserted into a living body; and
an alloy part provided in the elongated part and formed from an alloy containing titanium, tantalum, and tin, wherein
the alloy contains tantalum in an amount of 19 at. % or more and 27 at. % or less and tin in an amount of 2 at. % or more and 8 at. % or less relative to the whole alloy as 100 at. %, and the balance comprises titanium and an unavoidable impurity, and
the alloy part is provided to a portion to be inserted into a living body.

2. A medical instrument comprising:
an elongated part formed in a linear or tubular shape, at least a part of which is inserted into a living body; and
an alloy part provided in the elongated part and formed from an alloy containing titanium, tantalum, and tin, wherein
the alloy contains tantalum in an amount of 19 at. % or more and 27 at. % or less and tin in an amount of 2 at. % or more and 8 at. % or less relative to the whole alloy as 100 at. %, and the balance comprises titanium and an unavoidable impurity,
the alloy part is provided to a portion to be inserted into a living body, and
the alloy part is provided on a tip side of the elongated part.

3. The medical instrument according to claim 1, wherein the alloy part is formed in at least one shape selected from the group consisting of a spiral shape, a tubular shape, an annular shape, a cap shape, a tubular shape with a slit or a hole, a net shape, a basket shape, a linear shape, a rod shape, a flat plate shape, a curved plate shape, a needle shape, a needle tube shape, a columnar shape, and a block shape.

4. The medical instrument according to claim 2, wherein the alloy part is formed in at least one shape selected from the group consisting of a spiral shape, a tubular shape, an annular shape, a cap shape, a tubular shape with a slit or a hole, a net shape, a basket shape, a linear shape, a rod shape, a flat plate shape, a curved plate shape, a needle shape, a needle tube shape, a columnar shape, and a block shape.

5. The medical instrument according to claim 1, wherein the alloy part provides a predetermined first function and a function of radiographic visualization.

6. The medical instrument according to claim 5, wherein the first function is a reinforcing function for increasing a strength of the elongated part or suppressing deformation of the elongated part.

7. The medical instrument according to claim 5, wherein the first function is a rigidity adjusting function of adjusting axial rigidity, bending rigidity, or torsional rigidity of the elongated part.

8. The medical instrument according to claim 5, wherein the first function is a shaping function for causing the elongated part to have a predetermined bent shape.

9. The medical instrument according to claim 5, wherein the first function is a scraping function of scraping a part of a living body or an accretion to a living body.

10. The medical instrument according to claim 5, wherein the first function is a filter function for trapping a substance moving through a living body or filtering a fluid in a living body.

11. The medical instrument according to claim 5, wherein the first function is an ablation function of ablating a part of a living body or an accretion to a living body.

12. The medical instrument according to claim 5, wherein the first function is an anchoring function of anchoring to a living body.

13. The medical instrument according to claim 5, wherein the first function is a holding function of holding a part of a living body or an accretion to a living body.

14. The medical instrument according to claim 5, wherein the first function is a cutting function of cutting a part of a living body or an accretion to a living body.

15. The medical instrument according to claim 5, wherein the first function is a needling function of needling into a tissue of a living body.

16. The medical instrument according to claim 5, wherein the first function is a nozzle function for discharging a fluid into a living body.

17. The medical instrument according to claim 5, wherein the first function is a spiral propelling function for moving the elongated part in an axial direction by a rotation of the elongated part about the axial direction.

18. The medical instrument according to claim 5, wherein the first function is a passive function for moving the elongated part by a peristalsis of a living body.

19. The medical instrument according to claim 5, wherein the first function is an electrode function for passing an electric current through a living body or generating an electric field in a living body.

20. The medical instrument according to claim 5, wherein the first function is a length measurement function of measuring a length in a living body.
